(19) United States
(12) Patent Application Publication
(10) Pub. No.: US 2025/0128068 A1
(43) Pub. Date: Apr. 24, 2025

(54) SYSTEMS AND METHODS FOR STATE DETERMINATION AND NORMALIZATION USING SENSED SIGNALS

(71) Applicant: Boston Scientific Neuromodulation Corporation, Valencia, CA (US)

(72) Inventors: Lisa Denise Moore, Glendale, CA (US); G. Karl Steinke, Valencia, CA (US)

(21) Appl. No.: 18/904,394

(22) Filed: Oct. 2, 2024

Related U.S. Application Data
(60) Provisional application No. 63/544,978, filed on Oct. 20, 2023.

(51) Int. Cl. A61N 1/36; A61N 1/372

(52) U.S. Cl. CPC A61N 1/36139; A61N 1/37235

(57) ABSTRACT

Systems and methods for state determination and normalization using adaptive neuromodulation based on biopotentials and/or electrophysiology (e.g., evoked responses) are disclosed. An exemplary system comprises at least one lead, an electrostimulator to provide electrostimulation to a neural target, a sensing circuit to sense ERs to electrostimulation, and a controller circuit. In response to electrostimulation delivered to the neural target in accordance with a stimulation setting via a stimulating electrode, the controller circuit may collect sensed ERs to the electrostimulation using at least one sensing electrode. The controller circuit may determine at least one parameter associated with the electrostimulation or an affect of the electrostimulation to the neural target. The controller circuit may associate at least one feature of a first set of the sensed ERs with the parameter to classify the parameter.

### CLAIM OF PRIORITY

This application claims the benefit of U.S. Provisional Application No. 63/544,978, filed on Oct. 20, 2023, which is hereby incorporated by reference in its entirety.

### TECHNICAL FIELD

This document relates generally to medical systems, and more particularly, but not by way of limitation, to systems, devices, and methods for state determination and normalization using sensed signals.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc.

A therapy device may be configured to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. In another nonlimiting example, a device, such as a myocardial stimulator and/or neurostimulator, may be configured to treat cardiovascular condition. Settings of the therapy device may be programmed based on observed clinical effects so that the therapy provides desirable intended effects (e.g., reduced tremor, bradykinesia, and dyskinesia for a PD therapy, desirable pain relief or paresthesia coverage for a pain therapy, desirable blood pressure and/rhythms for a cardiovascular therapy) while avoiding undesirable side effects.

### SUMMARY

An example (e.g., “Example 1”) of a system may include an electrostimulator, a sensing circuit, and a controller circuit. The electrostimulator may be configured to provide electrostimulation to a neural target of a patient. The sensing circuit may be configured to sense an evoked response (ER) to the electrostimulation. The controller circuit may be operably connected to the electrostimulator and the sensing circuit. The controller circuit may be configured to determine at least one parameter associated with the electrostimulation or an affect of the electrostimulation to the neural target. The controller circuit may be configured to associate at least one feature of a first set of the sensed ERs with the at least one parameter to classify the parameter.

In Example 2, the subject matter of Example 1 may optionally be configured such that associating the at least one feature with the at least one parameter includes determining a relative or absolute state of the patient based on the at least one feature.

In Example 3, the subject matter of any one of Examples 1-2 may optionally be configured such that the controller circuit is configured to determine whether to perform an action based on the association of the at least one feature with the at least one parameter.

In Example 4, the subject matter of any one of Examples 1-3 may optionally be configured such that the at least one feature comprises at least one of a frequency of the sensed ERs, an amplitude of the sensed ERs, an area under curve, peak to peak differences, a latency or timing of one or more peaks, or relative measures of the same.

In Example 5, the subject matter of any one of Examples 1-4 may optionally be configured such that the controller circuit is further configured to determine a state of the patient based on whether a value for the at least one feature is within a defined range of values.

In Example 6, the subject matter of Example 5 may optionally be configured such that the state is determined to be a high state in response to the value being within a first threshold range of values; the state is determined to be a low state in response to the value being within a second threshold range of values; the state is determined to be an optimal state in response to the value being associated with a particular patient outcome; and the state is determined to be an average state in response to the value being within a third threshold range associated with a most common range of the at least one feature for a set period of time.

In Example 7, the subject matter of Example 6 may optionally be configured such that the high state, the low state, the optimal state, and the average state are each patient specific.

In Example 8, the subject matter of any one of Examples 1-7 may optionally be configured such that an inflection point of a set of values of the first set of sensed ERs that indicates a change in state; and a presence or location of the inflection point is used to determine the state.

In Example 9, the subject matter of any one of Examples 1-8 may optionally be configured such that the controller circuit is further configured to determine a medication state of the patient based on the at least one feature.

In Example 10, the subject matter of any one of claims 1-9 may optionally be configured such that the controller circuit is configured to determine, based on the at least one feature, a state of the patient from: a sleep state; an awake state; or a state of physical activity.

In Example 11, the subject matter of any one of Examples 1-10 may optionally be configured such that the controller circuit is further configured to determine a transition from a first state to a second state of the patient based on the at least one feature. The action may optionally include: an electrostimulation test; administering electrostimulation; adjusting delivery rate of medication; adjusting an amount of medication; or beginning an administration of medication.

In Example 12, the subject matter of any one of Examples 1-11 may optionally be configured such that the controller circuit is further configured to determine a state of the patient based on the association; collect additional sensed ERs to the electrostimulation; determine an additional state of the patient based on the additional sensed ERs; and in response to the determined state and the determined additional state being different, preventing comparison of the sensed ERs and the additional sense ERs.

In Example 13, the subject matter of any one of Examples 1-12 may optionally be configured such that the controller circuit is further configured to adjust a set of parameters associated with the electrostimulation based on the determined state of the patient; and adjustment of the set of parameters comprises adjusting one of a pulse amplitude, a pulse width, a pulse frequency, relative timing between areas or channels, an ON or OFF timing, stimulation patterns, pulse type, active electrodes, or fractionalization among active electrodes.

In Example 14, the subject matter of any one of Examples 1-13 may optionally be configured such that the controller circuit is further configured to estimate a disease severity of the patient based on the at least one feature.

In Example 15, the subject matter of any one of Examples 1-14 may optionally be configured such that the controller circuit is further configured to notify a physician to perform an assessment in response to the association being an association corresponding to performance of the assessment.

Example 16 includes subject matter (such as a method, means for performing acts, machine readable medium including instructions that when performed by a machine cause the machine to perform acts, or an apparatus to perform). The subject matter may include delivering electrostimulation to a neural target of a patient in accordance with a stimulation setting via a stimulating electrode selected from a plurality of electrodes on at least one lead. The subject matter may include sensing evoked responses (ERs) using sensing electrodes connected to a sensing circuit, the sensing electrodes selected from the plurality of electrodes on the at least one lead. A processing system may be used to determine at least one parameter associated with electrostimulation or an affect of the electrostimulation to the neural target. The processing system may be used to associate at least one feature of a first set of the sensed ERs with the at least one parameter to classify the at least one parameter.

In Example 17, the subject matter of Example 16 may optionally be configured such that associating the at least one feature with the at least one parameter comprises determining a relative or absolute state of the patient based on the at least one feature.

In Example 18, the subject matter of any of Examples 16-17 may optionally be configured such that a processing system is used to determine whether to perform an action based on the association of the at least one feature with the at least one parameter.

In Example 19, the subject matter of any of Examples 17-18 may optionally be configures such that at least one of a frequency of the sensed ERs, an amplitude of the sensed ERs, an area under curve, peak to peak differences, or a latency of one or more peaks.

In Example 20, the subject matter of any of Examples 16-19 may optionally be configured such the processing system is used to determine a state of the patient based on whether a value for the at least one feature is within a defined range of values.

In Example 21, the subject matter of Example 20 may optionally be configured such that the state is determined to be a high state in response to the value being within a first threshold range of values; the state is determined to be a low state in response to the value being within a second threshold range of values; the state is determined to be an optimal state in response to the value being associated with a particular patient outcome; and the state is determined to be an average state in response to the value being within a third threshold range associated with a most common range of the at least one feature for a set period of time.

In Example 22, the subject matter of Example 21 may optionally be configured such that the high state, the low state, the optimal state, and the average state are each patient specific.

In Example 23, the subject matter of any one of Examples 16-22 may optionally be configured such that the processing system is used to compare the at least one feature of the sensed ERs to a medication schedule associated with the patient; and determine a medication parameter of the patient. The medication parameter comprises one of: a shift in a medication schedule of the patient; a high medication state of the patient; or a decrease in a medication state of the patient.

In Example 24, the subject matter of any one of Examples 16-21 may optionally be configured such that the processing system is used to estimate a medication state of the patient by using a particular feature of the sensed ERs and a medication schedule of the patient.

In Example 25, the subject matter of Example 24 may optionally be configured such that the processing system is used to determine when a decrease in medication in the patient exceeds a particular medication threshold based on the association; and to determine when to adjust a medication schedule based on the estimated medication state and a previously determined medication schedule.

In Example 26, the subject matter of any one of Examples 16-25 may optionally be configured such that determining the at least one parameter of the patient comprises determining when a medication schedule has shifted.

In Example 27, the subject matter of any one of Examples 16-26 may optionally be configured such that determining the at least one parameter of the patient comprises determining an average pattern of response to medication administered to the patient.

In Example 28, the subject matter of any one of Examples 16-27 may optionally be configured such that the processing system is used to determine a state of the patient based on the association; compare the determined state of the patient to an expected state of the patient based on a time of medication dose taken; and in response to the determined state and the expected state being different, determine that an administration of the medication is being affected by an additional parameter.

In Example 29, the subject matter of any one of Examples 16-28 may optionally be configured such that the association indicates a disease severity of the patient. The processing system is used to, in response to the sensed ERs stabilizing faster, determining that the disease severity is low; and in response to the sensed ERs stabilizing slower, determining that the disease severity is high.

In Example 30, the subject matter of any one of Examples 16-29 may optionally be configured such that the association indicates an amount of brain fatigue of the patient. The processing system is used to, in response to the sensed ERs stabilizing faster, determining that the amount of brain fatigue is low; and in response to the sensed ERs stabilizing slower, determining that the amount of brain fatigue is high.

In Example 31, the subject matter of Example 30 may optionally be configured such that the processing system is used to: collect a first set of sensed ERs toward an earlier portion of a day; collect a second set of sensed ERs toward a later portion of a day; compare the first set to the second set; in response to the first set stabilizing faster and the second set stabilizing slower, determine that the amount of brain fatigue is high; and in response to the first set stabilizing slower and the second set stabilizing slower, determine that an amount of brain fatigue is low and a disease severity is high.

Example 32 includes subject matter (such as a method, means for performing acts, machine readable medium including instructions that when performed by a machine cause the machine to perform acts, or an apparatus to perform). The subject matter may include delivering electrostimulation to a neural target of a patient in accordance with a stimulation setting via a stimulating electrode selected from a plurality of electrodes on at least one lead. The electrostimulation may be delivered to the neural target of the patient via a medical-device system that comprises an electrostimulator and the at least one lead coupled thereto. The subject matter may include sensing evoked responses (ERs) using sensing electrodes connected to a sensing circuit. Instructions may be executable by a processor to determine at least one feature for a first set of the sensed ERs; determine a state of the patient based on the at least one feature; and determine whether to perform an action based on the state of the patient.

In Example 33, the subject matter of Example 32 may optionally be configured such that the instructions are executable by the processor to notify a physician to perform an assessment in response to the determined state being a state to use for performing the assessment.

In Example 34, the subject matter of any of Examples 32-33 may optionally be configured such that the instructions are executable by the processor to, in response to the determined state indicating an affect on the patient from medication is decreasing, adjust electrostimulation to compensate for the affect.

In Example 35, the subject matter of any one of Examples 32-34 may optionally be configured such that the instructions are executable by the processor to, in response to the determined state indicating a reduced response to therapy, set a limit on a rate of adjustment of the electrostimulation to avoid overtreatment.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7 illustrates, by way of example and not limitation, an example of a neuromodulation system configured to determine a state of a patient and normalize patient data using sensed signals.

FIG. 8A illustrates, by way of example and not limitation, a method of state determination and normalization using sensed signals.

FIG. 8B illustrates, by way of example and not limitation, a method of state determination and normalization using sensed signals.

FIG. 9 illustrates, by way of example and not limitation, a method of state determination and normalization using sensed signals.

FIG. 10 illustrates, by way of example and not limitation, a method of state determination and normalization using sensed signals.

FIG. 11 illustrates, by way of example and not limitation, an example of types of determined states using sensed signals for adjusting neuromodulation therapy.

FIG. 12 illustrates, by way of example and not limitation, an example of a number of actions based on determined states for adjusting neuromodulation therapy.

FIGS. 13A-13B illustrate, by way of example and not limitation, an example of distributions associated with evoked response features.

FIG. 14 illustrates, by way of example and not limitation, a neuromodulation therapy system, which may be used to deliver DBS.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

There may be differing effectiveness of neuromodulation therapy depending on a patient's state, e.g., vary depending on a medication level, a physical state (sleep, awake, physical activity, etc.), condition or disorder, anatomy (anatomical target), or symptoms to improve (e.g., tremor to cognitive skill improvement). States may also include an active state vs. inactive state, an ON or OFF state, low state, high state, optimal states, inflection points in state, stable states, transitioning from one state to another, etc. These states may be correlated to particular, or significant, events. The particular events may be used to normalize data recordings or to indicate optimal times for testing and/or providing therapy. Further, adjusting the neuromodulation therapy to these conditions may provide for more effective treatment.

State(s) of a patient can be determined based on electrophysiological responses, such as biopotentials, including evoked responses (ERs) and/or spontaneous response (SRs), including various frequency and magnitude scales, such as Evoked Potentials (EPs) and Local Field Potentials (LFPs). Evoked responses (ERs) may be used to determine these states or conditions of the patient. The programming of device settings (e.g., sensing parameters or neurostimulation parameters) may be adjusted and stored in relation to these states or conditions and used in subsequent administration of treatment in order to find more effective treatment more quickly. The ERs may be caused by stimulation that provides ERs, stimulation that provides therapy, and stimulation that may both deliver therapy and provide ERs. More specifically, features of the ERs may also be used to determine the state or condition of the patient. Examples of the signal features may include a signal amplitude, magnitude, peak value, value range, a signal curve length, or a signal power or RMS value of an ER signal within a time window, such as the epoch-averaged ERs, N1-P2 peak amplitude, N1-P2 delay (distance on the x-axis in recording time, which may be a measure of the period of decaying sine wave), size of evoked potential envelope, relative amplitudes of each peak (distance between any given peak and the midpoint). The amplitude can refer to a feature of an amount, a magnitude, a strength, etc. Further, a variety of ways to measure the amplitude can be used including peak to trough, envelope, squared, etc. Responses may be modulated by the details of the sensing, including amplifier settings, relationships between stimulating and sensing electrodes, natures of stimulating or sensing electrodes including geometry and surface among other factors, and signal processing occurring during and after measurement, including treatment within analogue or digital hardware, firmware, or software.

Features of the sensed signals may vary in relation to a medication state and may be used to estimate a schedule for administering medication and/or to estimate medication efficacy. Features of the sensed signals may be used to estimate a brain “age” and/or a disease severity. Features of the sensed signals may be used to estimate fatigue and/or brain fatigue. Sensed signals may be used to indicate to a physician (or determine as part of a system) when is the optimal time to perform particular assessments or provide particular therapy. States of a patient may be used to normalize data by comparing data associated with a particular state with data also associated with that particular state and not comparing data that is associated with a different state. Sensed signals may be used, in conjunction with one or more of the approaches mentioned herein, as a signal to maintain treatment stability within a set range of sensed signals.

This disclosure refers to an ERNA caused by electrostimulation, as a nonlimiting example of an ER to electrostimulation provided by an electrostimulator. The present subject matter may be applied for other ERs to other electrostimulation. The electrostimulation may be therapeutic in nature in some examples, or diagnostic in nature in others.

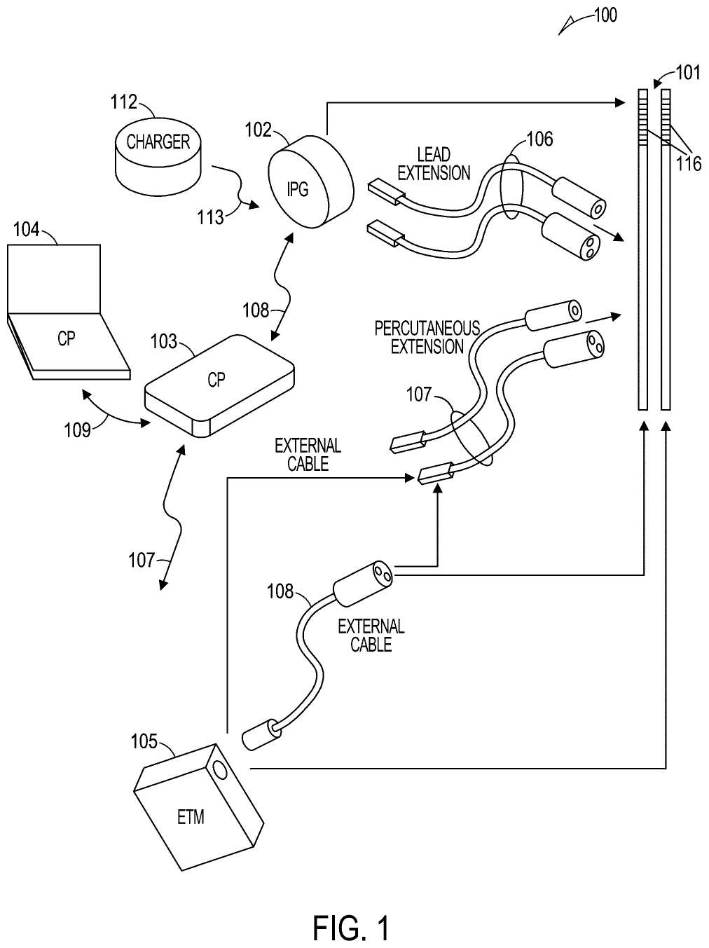

FIG. 1 illustrates, by way of example and not limitation, an electrical stimulation system 100, which may be used to deliver DBS. The electrical stimulation system 100 may generally include one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads may be provided, including only one, as long as the number of electrodes is greater than two (including the IPG case function as a case electrode) to allow for lateral steering of the current. Alternatively, a surgical paddle lead may be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 may likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters may be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies may be performed by executing software instructions contained within the CP 104. Alternatively, such programming methodologies may be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

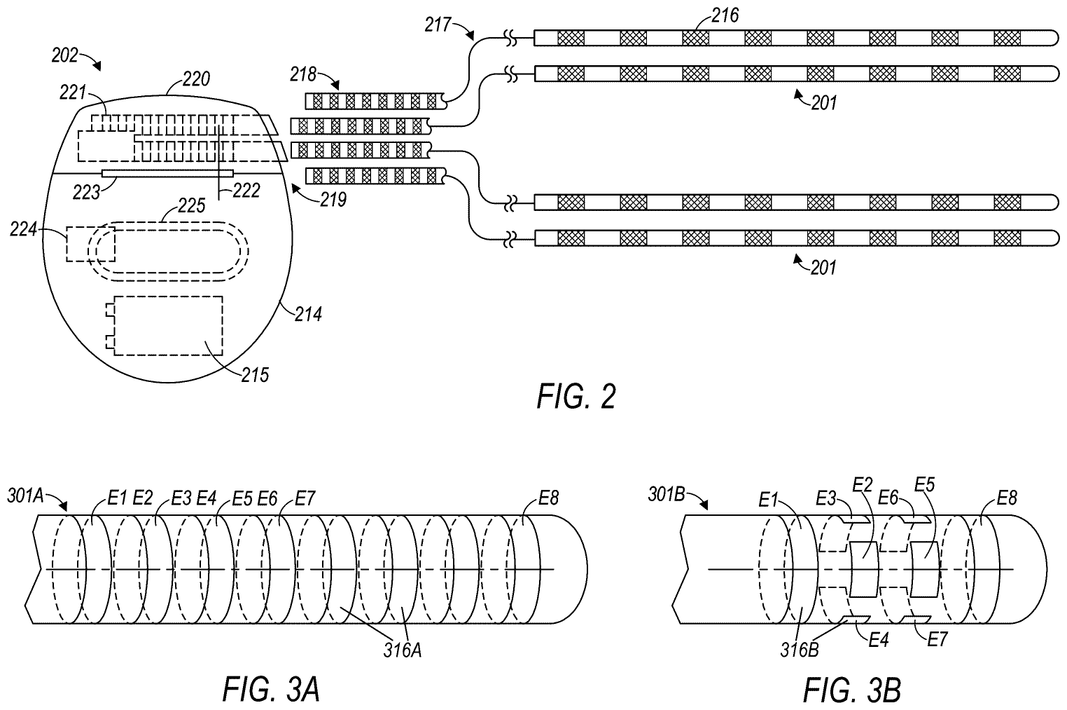

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more rows of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore may vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue; a comparison of a set threshold value; user-determined values or thresholds, or a change/delta from a prior measurement. Each of the electrodes may either be used (an active electrode) or unused (OFF). When the electrode is used, the electrode may be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a non-directional lead 301A with electrodes 316A disposed at least partially about a circumference of the non-directional lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a directional lead 301B with electrodes 316B including ring electrodes such as E1 at a proximal end and E8 at the distal end. Additionally, the lead 301 also includes a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide more-superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

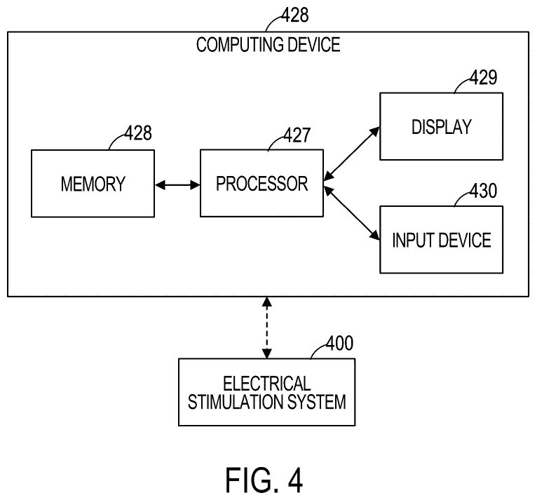

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease.

In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, a closed-loop algorithm for determining or updating stimulation parameters may be implemented in a mobile device, such as a smartphone, which is connected to the IPG or an evaluating device (e.g., a wristband or watch). These devices may also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and may include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

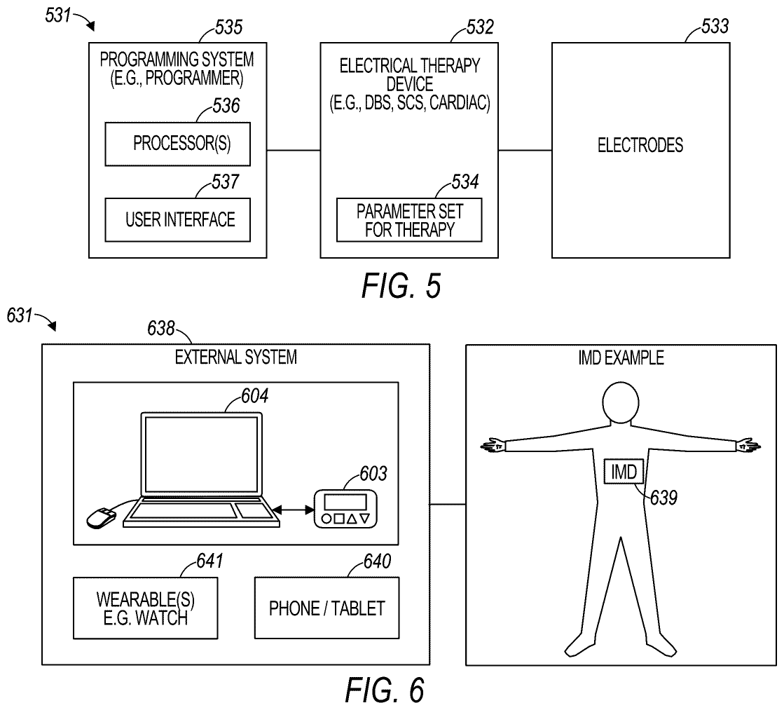

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameter sets through a computerized programming system to allow the optimum modulation parameters to be determined based on patient feedback or other means and to subsequently program the desired modulation parameter sets.

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

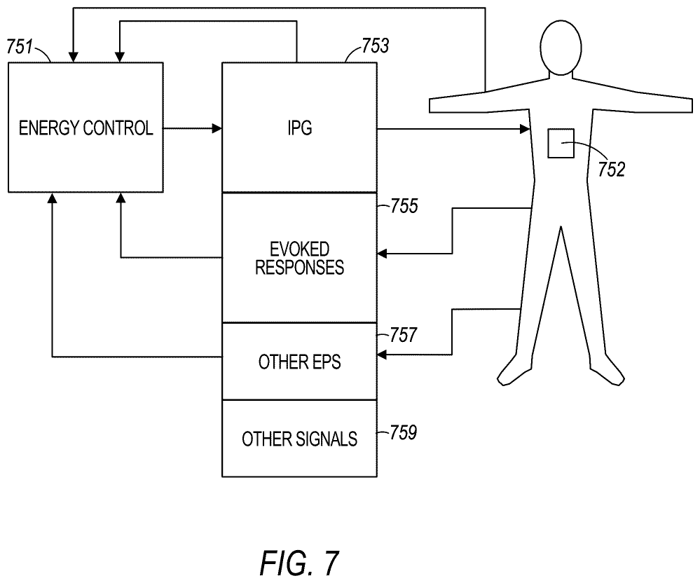

FIG. 7 illustrates, by way of example and not limitation, an example of a neuromodulation system configured to determine a state of a patient and/or normalize data using sensed signals to adjust neuromodulation therapy. The illustrated system in FIG. 7 includes components including an energy control unit 751, an implantable pulse generator (IPG) 753, evoked responses 755, other evoked potentials (EPs) 757, other signals 759, and an implantable medical device (IMD) 752 implanted within a patient. The IMD 752 may provide various ERs 755, and other signals 759 caused by various stimulation, sensors, etc., to provide for input to the energy control unit 751. The energy control unit 751 may be a device that includes hardware, software, and/or firmware used to process the received information and to provide instructions to the IPG 753 to provide neurostimulation to the patient through the IMD 752. The ERs 755, and other signals 759 may be used to determine a state of a patient, to normalize data, to provide various stimulation and neuromodulation therapy, among other approaches, as will be described herein, to provide effective therapy to the patient in a variety of different states and conditions.

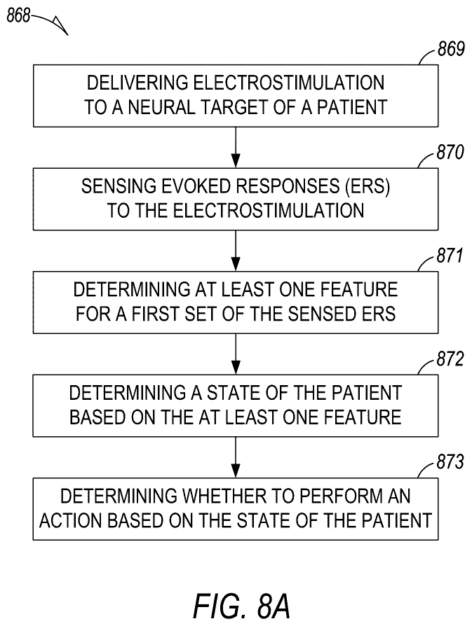

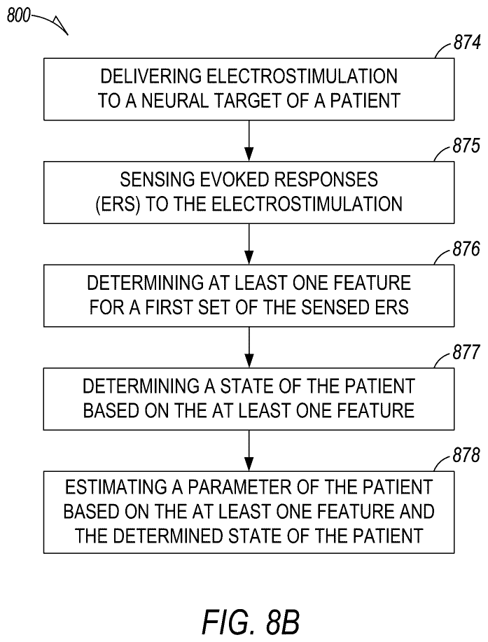

FIG. 8A illustrates, by way of example and not limitation, a method of state determination and normalization using sensed signals. At 869, the method 868 may include delivering electrostimulation to a neural target of a patient.

At 870, the method 868 may include sensing evoked responses (ERs) to the electrostimulation. Evoked responses (ERs) may be sensed using sensing electrodes connected to a sensing circuit. The sensing electrodes may be selected from the plurality of electrodes on the at least one lead. The ERs may be caused by stimulation that provides ERs, stimulation that provides therapy, and stimulation that may both deliver therapy and provide ERs.

At 871, the method 868 may include determining at least one feature for a first set of the sensed ERs. The at least one feature includes at least one of a frequency of the sensed ERs, an amplitude of the sensed ERs, an area under curve, peak to peak differences, or a latency of one or more peaks. In some examples, an inflection point of a set of values of the first set of sensed ERs indicates a local peak value that indicates a change in state. As an example, a point at which the set of values goes from an increasing set of values to a decreasing set of values may indicate a change in the state. As an example, a point at which the set of values goes from a decreasing set of values to an increasing set of values may indicate a change in the state. Further, a point at which the set of values goes from a constant set of values to a decreasing or increasing set of values may indicate a change in the state. A presence or location (e.g., a time of day, a location in the data such as at a start, middle, or end of the data, etc.) of the inflection point may be used to determine the state. The location of the inflection point can refer to a location within a series of values, a location in absolute time, and/or a location relative to some other factor or portion of data. Each of these locations may provide different approaches to determine what the inflection point is indicating. The inflection point can refer to a state of exercise, an inflection point based on a decrease or cessation of caffeine intake. The inflection point can be associated with changes in medication, food intake, physical activity, rest, mood, altitude, hydration, blood pressure, posture, etc. The inflection point may be associated with exhaustion or a level of exhaustion (e.g., tiredness), or alertness or a level of alertness. The inflection point may be associated with a wash-in or wash-out of stimulation.

At 872, the method 868 may include determining a state of the patient based on the at least one feature. The state of the patient may be determined based on whether a value for the at least one feature is within a defined range of values. The state may be determined to be a high state in response to the value being within a first threshold range of values. The state may be determined to be a low state in response to the value being within a second threshold range of values. The state may be determined to be an optimal state in response to the value being associated with a particular patient outcome. The state may be determined to be an average state in response to the value being within a third threshold range associated with a most common range of the at least one feature for a set period of time. The high state, the low state, the optimal state, and the average state may each be patient specific.

Based on the at least one feature, a state of the patient may be determined from a sleep state, an awake state, or a state of physical activity. The sleep state may be associated with a particular level of sleep, such as rapid eye movement (REM) sleep, or a particular stage of sleep (e.g., a first, second, third, or fourth stage of sleep). The awake state may be associated with a particular type of awake state such as an alert state, a focused state, a fatigued state, etc. The state of physical activity may be associated with a level of physical activity such as a low level, a medium level, and/or a high level that each indicate an intensity of activity being performed or recently performed by the body of the patient and/or other factors.

A medication state of the patient may be determined based on the at least one feature. As an example, a high medication state, a medium medication state, a low medication state, etc., may be determined based on the feature. The high medication state may indicate a high level of medication in the patient, a high absorption rate of the medication, and/or a high efficacy of the medication on the patient, where each of these examples indicates the patient's symptoms are highly alleviated or the medication is being more highly effective. The high level, absorption rate, effectiveness, etc., may be in relation to a medium or lower level, rate, or effectiveness based on many data points related to the patient. A medium medication state may indicate a medium level of medication in the patient, a medium absorption rate of the medication, and/or a medium efficacy of the medication on the patient, where each of these examples indicates the patient's symptoms are alleviated or the medication is effective to a moderate degree. The medium absorption rate, effectiveness, etc., may be in relation to a higher or lower rate or effectiveness based on many data points related to the patient. Likewise, the low medication state may indicate a low level of medication in the patient, a low absorption rate of the medication, and/or a low effectiveness of the medication on the patient, where each of these examples indicates the patient's symptoms are alleviated or the medication is being more effective to a lower degree than other levels, rates, or efficacies. The medication state can be an absolute medication state or a relative medication state. For example, the absolute medication state can be a medication state independent of other factors whereas a relative medication state can be a medication including comparisons of or effects from other factors. In some examples, the medication state may be considered on a continuum with extreme values being high and low and in-between states being calculated as between the high and low states.

Medication state may be estimated based on a patient's medication schedule or based on the exact times that patients are administered the medication. However, patients may forget to take the medication, may forget to take the medication using the correct medication schedule, may metabolize the medication at different rates (as the metabolization rates are patient specific), the medication may fail to have a proper effect (e.g., rendered ineffective), or absorption of the medication may be affected by dietary intake. For example, with respect to dietary intake, when the food is eaten, how much, what kind of food or drink was consumed may affect the medication. In addition, an affect of the medication can be altered based on a medication tablet failing to be processed in the stomach of the patient or absorbed in the stomach and/or intestines. The ER values or features in combination with the patient's previously determined or known medication schedule may help detect when a patient's medication has shifted. The ER values or features in combination may help detect when a patient achieves a maximal ON state after taking medication. The ER values or features in combination may help detect when medication is wearing off or being less effective to the patient. The ER values or features in combination may help detect an average pattern of response by the patient to the medication. Based on these estimates, patient specific medication metabolism rates may be estimated. Further, periods where wearing off exceeds a specific threshold may be identified. Medication schedule adjustments may be recommended based on these estimations. Anomalies in patient medication for the determined state compared to the time of dose taken may be used to identify factors affecting the medication or preventing the medication from taking effect or full effect. As an example, eating certain foods prior to taking medication may reduce the efficacy of certain medications.

The method 868 may include adjusting a set of parameters associated with the electrostimulation based on the determined state. For example, a first set of parameters may be used to perform electrostimulation in response to the patient being in the first state and a second set of parameters may be used when the patient is in a second state. The set of parameters may be adjusted to the first set or the second set, or an additional set, based on which state the patient is determined to be in. The adjustment of the set of parameters may include adjusting one of a pulse amplitude, a pulse width, a pulse frequency, relative timing between areas or channels, an ON or OFF timing, stimulation patterns, active electrodes, or fractionalization among active electrodes.

At 873, the method 868 may include determining whether to perform an action based on the state of the patient. The method 868 may include determining a transition from a first state to a second state based on the at least one feature. The action may include performing an electrostimulation test, administering electrostimulation, adjusting a delivery rate of medication, adjusting an amount of medication, or beginning an administration of medication.

The method 868 may include collecting additional sensed ERs to the electrostimulation, determining an additional state of the patient based on the additional sensed ERs, and, in response to the determined state and the determined additional state being different, preventing comparison of the sensed ERs and the additional sense ERs. When patient data is collected in a clinic or at home, it may be difficult to know how to compare data collected at two distinct time points. For example, data collected at a first state of medication should not be compared to data collected at a second state of medication. Similarly, data collected at different time points may reflect a more degenerated disease state and should not be compared. Data collected at different pulse widths may also not be compared due to the impact of pulse widths on outcomes being uncertain. By preventing comparison of sensed ERs recorded while the patient was in a different state, a normalization of the data may occur for the scenarios mentioned above. ER signals and/or feature(s) may be used to normalize data collected at different time points, under different medical conditions, at different wash in or wash out conditions, and/or different pulse width conditions, among other such varying conditions or scenarios. For example, more specifically, ER data recorded while a patient is in a first state may be compared with additional ER data recorded while the patient is in the same first state. ER data recorded while the patient is in a second state may be prevented from being compared to the ER data associated with the first state.

The method 868 may include estimating a disease severity of the patient based on the at least one feature. A disease severity can refer to the longevity, the duration, and/or a particular stage of the disease. A brain “age” of the patient may be estimated based on the at least one feature. For example, an ER signal and/or feature that stabilizes faster could indicate a younger or healthier (e.g., lower disease severity) versus a more slowly stabilizing ER signal and/or feature that may indicate an older or more severely diseased brain.

The method 868 may include estimating an amount of fatigue and/or brain fatigue of the patient based on the at least one feature. Fatigue can refer to a patient that has become tired, less responsive, is experiencing slower responses as compared to prior to fatigue, etc. Brain fatigue may refer to a patient that is experiences mental tiredness, is less mentally responsive, and is experiencing slower mental response as compared to prior to brain fatigue. Since there may be no way of normalizing fatigue states, changes in treatment may be avoided when fatigue is detected. In some examples, an ER signal and/or feature that stabilizes slower than normal may indicate brain fatigue. This may be distinguished by determining how quickly a patient stabilizes during an earlier period of time (e.g., during an earlier part of a day) and compare to whether the patient stabilizes more slowly at a later period of time (e.g., during a later part of the day), which could indicate fatigue. In this same example, if the patient stabilizes more slowly during both or many periods of time, it may indicate the above mentioned disease severity is greater. For example, brain fatigue may be distinguished from disease severity in that a younger but tired brain should be stabilizing quickly initially and then become slower throughout a period of time, through treatment withdrawal, or through constant treatment changes.

The method 868 may include notifying a physician to perform an assessment in response to the determined state being a state to use for performing the assessment. As an example, the ER signal and/or feature may be used to indicate to a physician (or determine as part of a larger system) an optimal (or appropriate) time or period of time to perform particular assessments and/or treatments. In some examples, the physician may want the patient to have a sufficient wash-in or decrease of effect from prior interventions. In some examples, the physician may want to ensure that the patient's stimulation and/or medication has washed into or affects have sufficiently increased to a maximal state in order to assess potential side affects and maximal treatment state(s). In some examples, the method 868 can include informing the patient to conduct an assessment. The patient may contact their physician and/or the patient may take further steps to get the assessment performed.

The method 868 may include maintaining treatment stability within a set range of ER values or at least one feature based on the determined at least one feature. The treatment stability may be maintained based on the at least one feature in addition to other parameters or factors. If a patient is at an inflection point in therapy and medication is decreasing or wearing off without an upcoming planned dose of medication, stimulation may be adjusted to compensate for the decrease in effect from the medication. If a patient is responding more slowly to the therapy, the system may set limits on internal adaptation to a slower rate to ensure that the patient is not being over-treated or going above an optimal treatment or effect and causing side effects.

FIG. 8B illustrates, by way of example and not limitation, a method of state determination and normalization using sensed signals. At 874, the method 800 may include delivering electrostimulation to a neural target of a patient. At 875, the method 800 may include sensing evoked responses (ERs) to the electrostimulation. Evoked responses (ERs) may be sensed using sensing electrodes connected to a sensing circuit. The sensing electrodes may be selected from the plurality of electrodes on the at least one lead. The ERs may be caused by stimulation that provides ERs, stimulation that provides therapy, and stimulation that may both deliver therapy and provide ERs.

At 876, the method 800 may include determining at least one feature for a first set of the sensed ERs. The at least one feature includes at least one of a frequency of the sensed ERs, an amplitude of the sensed ERs, an area under curve, peak to peak differences, or a latency of one or more peaks. In some examples, an inflection point of a set of values of the first set of sensed ERs indicates a local peak value that indicates a change in state. As an example, a point at which the set of values goes from an increasing set of values to a decreasing set of values may indicate a change in the state. As an example, a point at which the set of values goes from a decreasing set of values to an increasing set of values may indicate a change in the state. Further, a point at which the set of values goes from a constant set of values to a decreasing or increasing set of values may indicate a change in the state. A location (e.g., a time of day, a location in the data such as at a start, middle, or end of the data, etc.) of the inflection point and/or features captured from multiple sources including a patient or caregiver application may be used to determine the state.

The method 800 may include comparing the at least one feature of the sensed ERs to a medication schedule associated with the patient. The method 800 may include determining a medication parameter of the patient. The medication parameter may include one of a shift in a medication schedule of the patient, a high medication state of the patient, or a decrease in a medication state of the patient. The medication parameter can indicate whether proper medication or a proper amount of medication is being administered. The medication parameter can be an effectiveness of the medication or whether the medication is properly affecting the patient or providing alleviation of symptoms. The medication parameter can be used to determine whether to change the dosage amount and/or change the dosage schedule and/or frequency. A recommendation can be provided to add co-therapy (e.g., medication) alongside current stimulation.

At 877, the method 800 may include determining a state of the patient based on the at least one feature. The state of the patient may be determined based on whether a value for the at least one feature is within a defined range of values. The state may be determined to be a high state in response to the value being within a first threshold range of values. The state may be determined to be a low state in response to the value being within a second threshold range of values. The state may be determined to be an optimal state in response to the value being associated with a particular patient outcome. The state may be determined to be an average state in response to the value being within a third threshold range associated with a most common range of the at least one feature for a set period of time. The high state, the low state, the optimal state, and the average state may each be patient specific.

The method 800 may include adjusting a set of parameters associated with the electrostimulation based on the determined state. For example, a first set of parameters may be used to perform electrostimulation in response to the patient being in the first state and a second set of parameters may be used when the patient is in a second state. The set of parameters may be adjusted to the first set or the second set, or an additional set, based on which state the patient is determined to be in. The adjustment of the set of parameters may include adjusting one of a pulse amplitude, a pulse width, a pulse frequency, an ON or OFF timing, stimulation patterns, active electrodes, or fractionalization among active electrodes.

At 878, the method 800 may include estimating a parameter of the patient based on the at least one feature and the determined state of the patient. Estimating the parameter of the patient may include estimating a medication state by using a particular feature of the sensed ERs and a medication schedule of the patient. Estimating the parameter of the patient may include estimating a medication metabolism rate specific to the patient associated with the estimated medication state and a medication schedule of the patient. Estimating the parameter of the patient may include determining when a decrease in medication in the patient exceeds a particular medication threshold. Estimating the parameter of the patient may include determining when to adjust a medication schedule based on the estimated medication state and a previously determined medication schedule. Estimating the parameter of the patient may include determining when a medication schedule has shifted. Estimating the parameter of the patient may include determining an average pattern of response to medication administered to the patient.

The method 800 may include comparing the determined state of the patient to an expected state of the patient based on a time of medication dose taken. In response to the determined state and the expected state being different, an affect on the administration of the medication by an additional parameter may be determined. In some examples, the estimated parameter may be a disease severity of the patient. In response to the sensed ERs stabilizing faster, a determination that the disease severity is low may be made. In response to the sensed ERs stabilizing slower, a determination that the disease severity is high may be made.

In some examples, the estimated parameter may be an amount of brain fatigue of the patient. In response to the sensed ERs stabilizing faster, a determination that the amount of brain fatigue is low may be made. In response to the sensed ERs stabilizing slower, a determination that the amount of brain fatigue is high may be made. The method 800 may include collecting a first set of sensed ERs toward an earlier portion of a day. A second set of sensed ERs may be collected toward a later portion of a day. A comparison of the first set to the second set may be performed. In response to the first set stabilizing faster and the second set stabilizing slower, a determination that the amount of brain fatigue is high may be made. In response to the first set stabilizing slower and the second set stabilizing slower, a determination that an amount of brain fatigue is low and a disease severity is high may be made. In this way, a distinction between disease severity and brain fatigue may be made and a severity of the disease and/or whether brain fatigue is present may be determined.

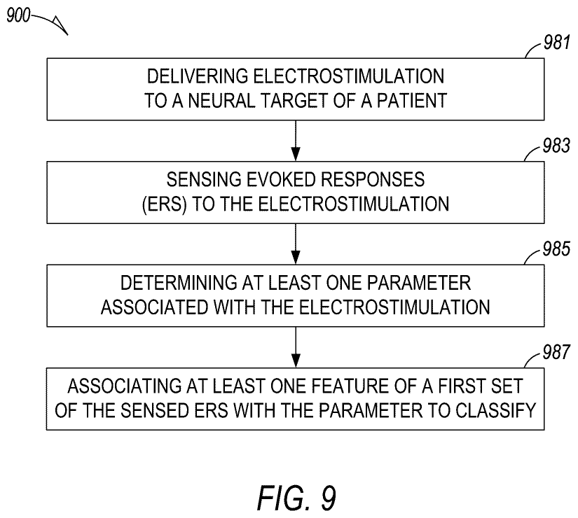

FIG. 9 illustrates, by way of example and not limitation, a method of state determination and normalization using sensed signals. At 981, the method 900 includes delivering electrostimulation to a neural target of a patient in accordance with a stimulation setting via a stimulating electrode.

At 983, the method 900 includes sensing evoked responses (ERs) to the electrostimulation using at least one sensing electrode.

At 985, the method 900 includes determining at least one parameter associated with the electrostimulation or an affect of the electrostimulation to the neural target. Determining the at least one parameter of the patient may include determining when a medication schedule has shifted. Determining the at least one parameter of the patient may include determining an average pattern of response to medication administered to the patient.

At 987, the method 900 includes associating at least one feature of a first set of the sensed ERs with the parameter to classify the parameter. Associating the at least one feature with the parameter may include determining a relative or absolute state of the patient based on the at least one feature. In some examples, the method 900 may include determining whether to perform an action based on the association of the at least one feature with the parameter. The at least one feature may include at least one of a frequency of the sensed ERs, an amplitude of the sensed ERs, an area under curve, peak to peak differences, or a latency of one or more peaks.

The at least one feature of the ERs can be associated with other parameters in order to differentiate among a scale, normalization, striation, ordering, classification, etc. of the treatment and/or therapy values. For example, as is described herein, the features of the ERs can be used to determine whether to be compared to one another (based on parameters that indicate that values obtained have similar enough characteristics to be comparable, such as recorded same time of day, same stage of medication, same therapy response, etc.). Further as an example, the ERs may be paired to Clinical Effects Scores (CES) entered into the Clinical Programmer (CP 104 in FIG. 1). CESes may refer to a score that measures the clinical effects of treatment (e.g., medication or stimulation) and/or other stimuli. Clinical effects may include therapeutic effects and/or side effects, or motor symptoms such as bradykinesia, tremor, or rigidity, and a clinical response may be evaluated based on the detected clinical effects.

In some examples, two same-value CESs having different ERs can be used for a first approach or treatment paradigm. Two different CESs having the same ERs may use a second, different approach or treatment paradigm (whereas a downstream approach treats the scores differently). And two CES values that are the same and are also associated with the same ERs may have a third approach or treatment paradigm. In this way, the different parameters, such CES in this example, can be used to compare and/or normalize the data for subsequent treatment approaches.

The association of the at least one feature with the parameter may be used to indicate a disease severity of the patient. The method 900 may include, in response to the sensed ERs stabilizing faster, determining that the disease severity is low, and, in response to the sensed ERs stabilizing slower, determining that the disease severity is high. The association may indicate an amount of brain fatigue of the patient. The method 900 can include, in response to the sensed ERs stabilizing faster, determining that the amount of brain fatigue is low, and, in response to the sensed ERs stabilizing slower, determining that the amount of brain fatigue is high.

The method 900 may include collecting a first set of sensed ERs toward an earlier portion of a day and collecting a second set of sensed ERs toward a later portion of a day. The method 900 may further include comparing the first set to the second set and, in response to the first set stabilizing faster and the second set stabilizing slower, determining that the amount of brain fatigue is high. In response to the first set stabilizing slower and the second set stabilizing slower, a determination that an amount of brain fatigue is low and a disease severity is high can be made.

In some examples, the method 900 may include determining a state of the patient based on whether a value for the at least one feature is within a defined range of values. The state of the patient may be determined to be a high state in response to the value being within a first threshold range of values. The state of the patient may be determined to be a low state in response to the value being within a second threshold range of values. The state of the patient may be determined to be an optimal state in response to the value being associated with a particular patient outcome. The state of the patient may be determined to be an average state in response to the value being within a third threshold range associated with a most common range of the at least one feature for a set period of time. The high state, the low state, the optimal state, and the average state may each patient specific.

In some examples, the method 900 may include comparing the at least one feature of the sensed ERs to a medication schedule associated with the patient and determining a medication parameter of the patient. The medication parameter may include one of: a shift in a medication schedule of the patient; a high medication state of the patient; or a decrease in a medication state of the patient. In some examples, the method 900 may include estimating a medication state of the patient by using a particular feature of the sensed ERs and a medication schedule of the patient. A decrease in medication in the patient may be determined when the medication exceeds a particular medication threshold based on the association. A determination of when to adjust a medication schedule may be based on the estimated medication state and a previously determined medication schedule. In some examples, the method 900 may include determining a state of the patient based on the association. The method 900 may include comparing the determined state of the patient to an expected state of the patient based on a time of medication dose taken. The method 900 may include, in response to the determined state and the expected state being different, determining that an administration of the medication is being affected by an additional parameter.

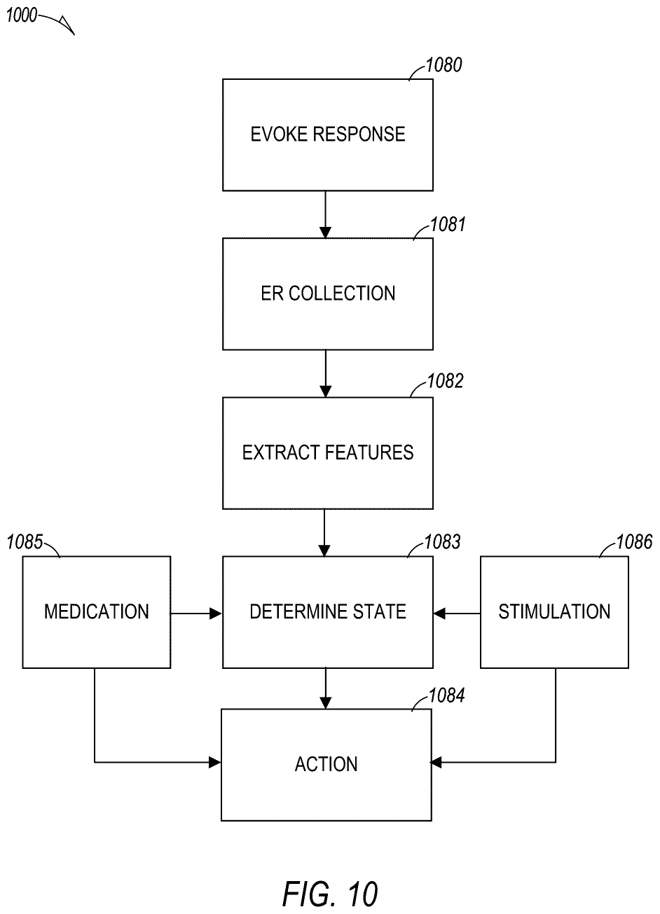

FIG. 10 illustrates, by way of example and not limitation, a flowchart of a method of state determination and normalization using sensed signals. At 1080, the flowchart 1000 may include evoking a response. The response, e.g., evoked response (ER), may be evoked by delivering electrostimulation to a neural target of a patient in accordance with a stimulation setting. The electrostimulation may be delivered via a stimulating electrode. The stimulation electrode may be selected from a plurality of electrodes on at least one lead. The electrostimulation may be delivered to the neural target of the patient via a medical-device system that may include an electrostimulator and the at least one lead coupled thereto. Responses may be modulated by the details of the sensing, as is described below, including amplifier settings, relationships between stimulating and sensing electrodes, natures of stimulating or sensing electrodes including geometry and surface among other factors, and signal processing occurring during and after measurement, including treatment within analogue or digital hardware, firmware, or software.

At 1081, the flowchart 1000 may include collecting ERs. ERs may be sensed using sensing electrodes connected to a sensing circuit. The sensing electrodes may be selected from the plurality of electrodes on the at least one lead. The ERs may be caused by stimulation that provides ERs, stimulation that provides therapy, and stimulation that may both deliver therapy and provide ERs.

At 1082, the flowchart 1000 may include extracting features (or at least one feature) from the collected ERs. The at least one feature includes at least one of a frequency of the sensed ERs, an amplitude of the sensed ERs, an area under curve, peak to peak differences, or a latency of one or more peaks.

At 1083, the flowchart 1000 may include determining a state of a patient. The state may be determined to be a high state, a low state, an optimal state, or an average state, as described above in association with FIGS. 8A-8B. Each of the states may be patient specific. The state of the patient may be determined from a sleep state, an awake state, a state of physical activity, etc.

The state may be determined based on a plurality of other factors and/or data inputs. For example, medication data 1085 may be used to determine the state of the patient. The medication data may affect the determination of the state of the patient. For example, if the medication level in the patient is high, the state of the patient may be different than if the medication level is low, etc. Likewise, stimulation data 1086 may affect the determination of the state of the patient. For example, if the stimulation level is high in the patient, the state of the patient may be different than if the stimulation level is low, etc.

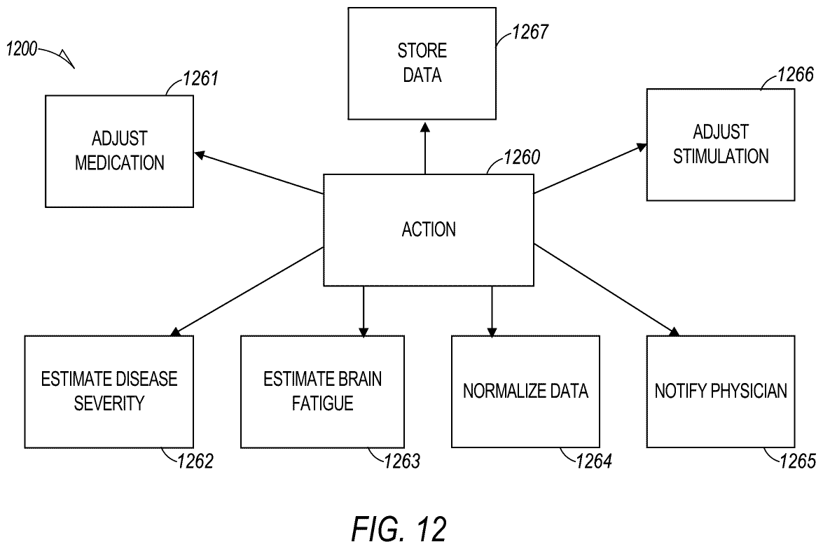

At 1084, an action may be performed based on the determined state. As is illustrated in FIG. 12, an action may include adjusting medication, estimating a disease severity of the patient, estimating brain fatigue, normalizing data, notifying a physician, adjusting stimulation, and/or storing data for subsequent or future analysis, reference, monitoring and/or display.

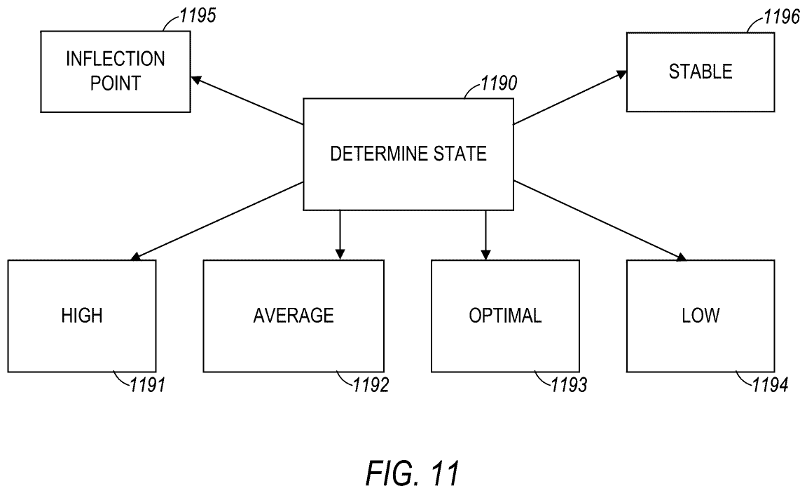

FIG. 11 illustrates, by way of example and not limitation, an example of types of determined states using sensed signals for adjusting neuromodulation therapy. The determined states 1190 may include a high state 1191, an average state 1192, an optimal state 1193, a low state 1194, an inflection point 1195, or a stable state 1196. The high state 1191 and low state 1194 may indicate that the state of the patient is above or below a threshold value in relation to medication, stimulation, among other factors. The high 1191 and low 1194 states may be used to determine if a value should be decreased or increased, or whether a parameter should begin or stop, such as administration of medication or stimulation. The high 1191 and low 1194 may be established as the highest or lowest range of the ER values or features, recorded as a set percentage of the time, or over a set period of time. The average state 1192 may indicate the most common range of the ER value or features over a set period of time. The optimal state 1193 may be the range of the ER values or features associated with the best patient outcomes or desired effects once a threshold amount of outcomes data has been collected. Outcomes data may include motor or non-motor data collected actively or passively through qualitative or quantitative means.

An inflection point 1195 in the data may be used to find local peaks or changes in the state of the patient. In some examples, the ER values or features and the state are not continuously variable. For example, there is a transition in ER-derived features which indicate the patient is transitioning from one state to another. The “location” of the transition (e.g., amplitude of evoking stimulation) may be used to titrate the state. The state transition may be periodically re-established and/or re-queried. The state, transitions, and/or control signals or features may differ between conditions, sub-types, and/or symptoms. In some examples, the ER values or measurements need to be queried to ensure that the patient is in the correct state to be measured. For example, an allowed time to wash-in or wash-out medication or to allow for verification of measurement while the DBS is in an ON or OFF condition. The state determination may affect stimulation adjustments such as amplitude, rate (up or down) or field movement. In some examples, particular patient states (such as sleep/awake, rested/fatigued, active or restful) could be known or controlled in order to modulate the responses.

FIG. 12 illustrates, by way of example and not limitation, an example of a number of actions based on determined states for adjusting neuromodulation therapy. The number of actions 1260 may include adjusting medication 1261, estimating disease severity 1262, estimating brain fatigue 1263, normalizing data 1264, notifying a physician 1265, adjusting stimulation 1266, or storing data 1267, among other actions not described herein. Whether to perform each or some of the actions 1260 may be determined based on a need of a patient. For example, when a patient is experiencing particular symptoms, or medication or stimulation is having less of an effect, adjusting medication 1261 or adjusting stimulation 1266 may be performed. Adjusting medication 1261 or adjusting stimulation 1226 may be performed to decrease the symptoms or increase an effect on the symptoms by the treatment.

When data is being processed and comparison are being made in order to determine next steps or additional treatment setup, normalizing data 1264 may be performed in order to compare the proper data sets to determine how to best administer treatment or therapy. In response to a patient experiencing an emergency or in response to the patient being in a state to have a test performed or particular therapy administered, a physician may be notified 1265 in order to administer the therapy or perform the appropriate tests. Further, when the effect of therapy is diminished, a disease severity 1262 or brain fatigue 1263 may be estimated or the distinction between severity or fatigue may be determined. In this way, the determined state of the patient may determine which of the actions 1260 to perform in order to properly address the patient in the state that the patient is in. Data (e.g., patient data, medication data, stimulation data, patient state data, ER data, feature data, etc.) may be stored 1267 in order to analyze the data or for future reference. The data may be stored 1267 for monitoring or display purposes.

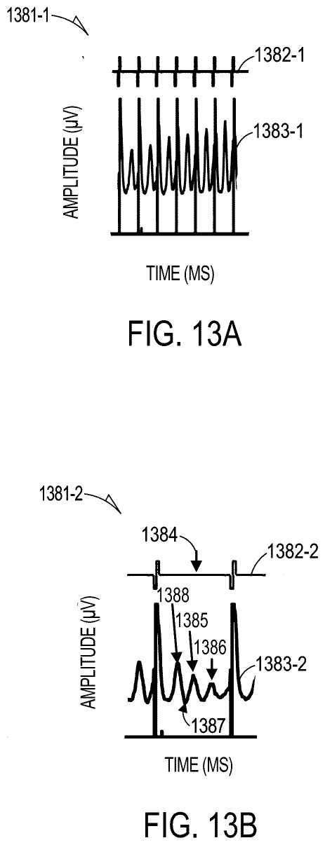

FIGS. 13A-13B illustrate, by way of example and not limitation, an example of distributions associated with evoked response features. The distributions 1381-1, 1381-2 each illustrate an affect that a corresponding generated stimulation 1382-1, 1382-2 has on the patient. For example, FIG. 13A illustrates that the stimulation 1382-1 causes ER signals 1383-1 where each subsequent secondary amplitude peak between primary amplitude peaks of the ER signals 1383-1 are steadily increasing in response to each pulse of the stimulation, showing a building or summation effect. The primary amplitude peak refers to a peak caused in close proximity to the stimulation and a secondary amplitude peak refers to a peak due to a lagging effect of the ER signal behavior. These secondary amplitude peak increases may build in response to the pulses being a particular distance from a previous and subsequent pulse, thereby causing a higher and higher amplitude peak per pulse, as shown. Likewise, at the cessation of the pulses, the amplitude peaks and overall amplitude of the ER signals 1383-1 quickly decline. These secondary amplitude peaks may be at least one ER feature used to determine a state or condition of the patient.

FIG. 13B illustrates an example where a gap or time delay 1384 between stimulation pulses of the stimulation 1382-2 causes a lagging decay effect in the ER signals 1383-2. For example, the secondary amplitude peaks 1385 and 1386 show a decline in amplitude peak over time as the time delay 1384 between stimulation pulses is increased. This decay effect may be an ER feature used to determine a state or condition of the patient for purposes of neuromodulation therapy.

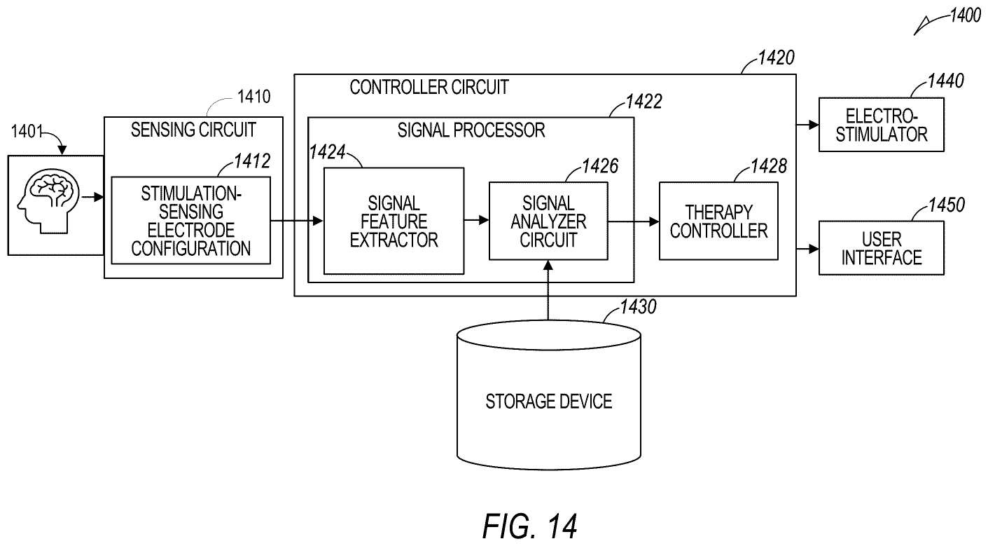

FIG. 14 illustrates, by way of example and not limitation, a neuromodulation therapy system, which may be used to deliver DBS based on evoked responses. The system 1400 includes a sensing circuit 1410, a controller circuit 1420, a storage device 1430, an electrostimulator 1440, and a user interface 1450. Portions of the system 1400 may be implemented in the IPG 102 or the CP 104.

The sensing circuit 1410 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 1440. The ring electrodes and/or the segmented electrodes may be configured as sensing electrodes for sensing ERs, or as stimulating electrodes for delivering electrostimulation pulses. The sensing circuit 1414 may sense ERs from one or more sensing electrodes on a lead placed at target issue (e.g., STN) of a patient 1401 in response to electrostimulation pulses delivered from a stimulating electrode at a stimulation site (e.g., a brain target). The ERs may be sensed in accordance with a stimulating-sensing electrode configuration 1412.

The controller circuit 1420 may include circuit sets comprising one or more other circuits or sub-circuits, such as a signal processor 1422 and a therapy controller 1428. The signal processor 1422 may further include a signal feature extractor 1424 and a signal analyzer circuit 1426. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 1420 may be implemented as a part of a microprocessor circuit. The microprocessor circuit may be a dedicated processor such as a digital signal processor, application specific integrated circuit (ASIC), microprocessor, or other type of processor for processing information including physical activity information. Alternatively, the microprocessor circuit may be a general purpose processor that may receive and execute a set of instructions of performing the methods or techniques described herein.

The signal feature extractor 1424 may extract a signal feature from the filtered ER signal (e.g., the ER signal with the stimulation artifact removed). Examples of the signal features may include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, may be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1-P2 P2P” amplitude). The signal curve length may be measured as accumulated signal value differences of the sensed evoked response (or an epoch-averaged evoked response) over consecutive unit times (e.g., consecutive data sampling intervals) within the time window. The signal power may be measured as an area under the curve (AUC) of the sensed evoked response (or the epoch-averaged evoked response) within the time window. In some examples, the signal analyzer circuit 1426 may generate a spatial distribution of extracted signal features across sensing locations of the sensing electrodes.

The signal analyzer circuit 1426 may compare the filtered ERs, or signal features or a spatial distribution of signal features derived therefrom, to one or more states or conditions of the patient. In some examples, the states or conditions are used to adjust the neuromodulation parameters. In some examples, the signal analyzer circuit 1426 may accumulate the sensed ERs, and therefore determined states or conditions, obtained in multiple stimulation-ER recording sessions during which stimulation pulses are delivered via a particular stimulating electrode with varying stimulation parameter settings (e.g., stimulation amplitude, frequency, or pulse width), and compare the accumulated ERs (or signal features or a spatial distribution of signal features derived from the filtered ERs) to previously stored states or conditions. In an example, the states or conditions of the patient may be user-provided or loaded into the system for easy recognition. In another example, the state or condition may be associated with a target ER or target ER feature template representing a patient-specific ER feature or a population-based ER feature to electrostimulation of the neural target. In an example, the target ER template may be used to adjust the neuromodulation therapy to relieve symptoms or other goals such as co-therapy (e.g., leads that inject drugs or light), and side-effect avoidance. The signal analyzer circuit 1426 may determine a distribution of sensed ER features, compare the determined distribution of sensed ERs to the corresponding states or conditions to determine whether to adjust the neuromodulation therapy.

The therapy controller 1428 may generate a control signal to the electrostimulator 1440 to adjust the thresholds of the neuromodulation therapy based on the determined state or condition. The electrostimulator 1440 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 1428 may be implemented as a proportional integral (PI) controller, a proportional-integral-derivative (PID) controller, or other suitable controller that takes the comparison of the sensed ERs (or features or a distribution of the features thereof) to the corresponding states or conditions as a feedback on the adjustment of stimulation settings.

The electrostimulator 1440 may be an implantable module, such as incorporated within the IPG 102 in FIG. 1. Alternatively, the electrostimulator 1440 may be an external stimulation device, such as incorporated with the ETS 40. In some examples, the user may choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 1440 may deliver stimulation in accordance with the adjusted therapy setting.

In some examples, the therapy controller 1428 may generate a recommendation to the user to adjust the device setting (e.g., a programmable parameter of the electrostimulator 1440) to cause the sensed ERs to align with or to compare more favorably to one or more states or conditions of the patient. In some embodiments, the display may provide a suggestion to the user to adjust stimulation parameters to cause the since developed responses to more favorably compare to the state or condition or a transition to a different state or condition. In some examples, the therapy controller 1428 may determine or modify therapeutic stimulation settings based on the sense ERs or features thereof and the corresponding determined state or condition of the patient. The electrostimulator 1440 may deliver therapeutic stimulation (e.g., DBS) in accordance with the determined or modified therapeutic stimulation settings.

In some examples, the user interface 1450 allows a physician to remotely review therapy settings and treatment history, consult with the patient to obtain information including pain relief and SCS-related side effects or symptoms, perform remote programming of the electrostimulator 1440, or provide other treatment options to the patient. The user interface 1450 may allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program may include a set of stimulation parameters with respective pre-determined values. In some examples, the user interface 1450 may include a display to display textually or graphically information provided by the user via an input unit, and device settings including, for example, feature selection, sensing configurations, signal pre-processing settings, therapy settings, optionally with any intermediate calculations. In an example, the user interface 1450 may present to the user an “optimal” or improved therapy setting, such as determined based on a closed-loop or adaptive feedback control of electrostimulation based on a selected evoked response signal feature, in accordance with various embodiments discussed in this document. In some examples, the user may use the user interface 1450 to provide feedback on a neuromodulation therapy, including, for example, side effects or symptoms arise or persist associated with the neurostimulation, or severity of the symptom or a side effect.

As used herein, the term “machine readable medium” may include any medium that is capable of storing, encoding, or carrying instructions for execution by a processing device or machine and that causes the processing device or machine to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method, comprising:
delivering electrostimulation to a neural target of a patient in accordance with a stimulation setting via a stimulating electrode;
sensing evoked responses (ERs) to the electrostimulation using at least one sensing electrode;
determining at least one parameter associated with the electrostimulation or an affect of the electrostimulation to the neural target; and
associating at least one feature of a first set of the sensed ERs with the at least one parameter to classify the at least one parameter.

2. The method of claim 1, wherein associating the at least one feature with the at least one parameter comprises determining a relative or absolute state of the patient based on the at least one feature.

3. The method of claim 1, comprising determining whether to perform an action based on the association of the at least one feature with the at least one parameter.

4. The method of claim 1, wherein the at least one feature comprises at least one of a frequency of the sensed ERs, an amplitude of the sensed ERs, an area under curve, peak to peak differences, or a latency of one or more peaks.

5. The method of claim 1, comprising determining a state of the patient based on whether a value for the at least one feature is within a defined range of values.

6. The method of claim 5, wherein:
the state is determined to be a high state in response to the value being within a first threshold range of values;
the state is determined to be a low state in response to the value being within a second threshold range of values;
the state is determined to be an optimal state in response to the value being associated with a particular patient outcome; and
the state is determined to be an average state in response to the value being within a third threshold range associated with a most common range of the at least one feature for a set period of time.

7. The method of claim 6, wherein the high state, the low state, the optimal state, and the average state are each patient specific.

8. The method of claim 1, comprising:
comparing the at least one feature of the sensed ERs to a medication schedule associated with the patient; and
determining a medication parameter of the patient;
wherein the medication parameter comprises one of:
a shift in a medication schedule of the patient;
a high medication state of the patient; or
a decrease in a medication state of the patient.

9. The method of claim 1, comprising estimating a medication state of the patient by using a particular feature of the sensed ERs and a medication schedule of the patient.

10. The method of claim 9, comprising:
determining when a decrease in medication in the patient exceeds a particular medication threshold based on the association; and
determining when to adjust a medication schedule based on the estimated medication state and a previously determined medication schedule.

11. The method of claim 1, wherein determining the at least one parameter of the patient comprises determining when a medication schedule has shifted.

12. The method of claim 1, wherein determining the at least one parameter of the patient comprises determining an average pattern of response to medication administered to the patient.

13. The method of claim 1, comprising:
determining a state of the patient based on the association;
comparing the determined state of the patient to an expected state of the patient based on a time of medication dose taken; and
in response to the determined state and the expected state being different, determining that an administration of the medication is being affected by an additional parameter.

14. The method of claim 1, wherein:
the association indicates a disease severity of the patient;
the method comprises:
in response to the sensed ERs stabilizing faster, determining that the disease severity is low; and
in response to the sensed ERs stabilizing slower, determining that the disease severity is high.

15. The method of claim 1, wherein:
the association indicates an amount of brain fatigue of the patient;
the method comprises:
in response to the sensed ERs stabilizing faster, determining that the amount of brain fatigue is low; and
in response to the sensed ERs stabilizing slower, determining that the amount of brain fatigue is high.

16. The method of claim 15, comprising:
collecting a first set of sensed ERs toward an earlier portion of a day;
collecting a second set of sensed ERs toward a later portion of a day;
comparing the first set to the second set;
in response to the first set stabilizing faster and the second set stabilizing slower, determining that the amount of brain fatigue is high; and
in response to the first set stabilizing slower and the second set stabilizing slower, determining that an amount of brain fatigue is low and a disease severity is high.

17. A non-transitory computer-readable medium storing instructions executable by a processor to:
deliver electrostimulation to a neural target of a patient, wherein the electrostimulation is delivered to the neural target of the patient via a medical-device system that comprises an electrostimulator;
sense evoked responses (ERs) to the electrostimulation using sensing electrodes connected to a sensing circuit;
determine at least one feature for a first set of the sensed ERs;
determine a state of the patient based on the at least one feature; and
determine whether to perform an action based on the state of the patient.

18. The non-transitory computer-readable medium of claim 17, wherein the instructions are executable by the processor to notify a physician to perform an assessment in response to the determined state being a state to use for performing the assessment.

19. The non-transitory computer-readable medium of claim 17, wherein the instructions are executable by the processor to:
in response to the determined state indicating an affect on the patient from medication is decreasing, adjusting electrostimulation to compensate for the affect; or
in response to the determined state indicating a reduced response to therapy, setting a limit on a rate of adjustment of the electrostimulation to avoid overtreatment.

20. A system, comprising:
an electrostimulator configured to provide electrostimulation to a neural target of a patient;
a sensing circuit configured to sense evoked responses (ERs) to the electrostimulation; and
a controller circuit operably connected to the electrostimulator and the sensing circuit, the controller circuit configured to:
determine at least one parameter associated with the electrostimulation or an affect of the electrostimulation to the neural target; and
associate at least one feature of a first set of the sensed ERs with the at least one parameter to classify the at least one parameter.
